(19) United States
(12) Patent Application Publication
(10) Pub. No.: US 2017/0042593 A1
(43) Pub. Date: Feb. 16, 2017

(54) MEDICAL IMPLANT

(71) Applicant: INVIBIO LIMITED, Lancashire (GB)

(72) Inventors: Christopher Richard George NEWMAN, Liverpool (GB); Andrew James ANDERSON, Lancashire (GB); Steven LAMORINIERE, Chorley (GB); Matthew CANTWELL, Shaker Heights, OH (US)

(73) Assignee: INVIBIO LIMITED, Lancashire (GB)

(21) Appl. No.: 15/118,963

(22) PCT Filed: Feb. 17, 2015
(86) PCT No.: PCT/GB2015/050451

§ 371 Date: Aug. 15, 2016

(30) Foreign Application Priority Data
Feb. 20, 2014 (GB) 1403032.4

(51) Int. Cl. A61B 17/72; A61B 17/80; A61L 31/12; A61L 31/18; A61L 31/06

(52) U.S. Cl. CPC A61B 17/7241; A61L 31/18; A61L 31/06; A61L 31/126; A61B 17/80; A61L 2430/02

(57) ABSTRACT

An IM nail is positioned within a medullary canal and secured in position by bone screws. The nail includes bores which engage insert assemblies and engage the screws. Each insert assembly is in two parts. Part includes a head and a narrower neck; and similarly, part includes a head and a narrower neck. A bore extends through the assembly from one side to the other. Part of the assembly is engaged with a nail by insertion into the nail via mouth; and part is engaged with the nail by insertion into the nail via mouth. When in position the parts abut one another and define bore which can receive screws. The nail is made from a polyetheretherketone (PEEK)/carbon fibre composite. Parts of the insert assemblies are produced separately from the nail. They may be produced by injection moulding a polymeric composition comprising PEEK and barium sulphate.

The invention relates to a medical implant and particularly, although not exclusively, relates to a medical implant which comprises an intra-medullary nail or rod or a bone plate.

An intra-medullary (IM) nail is used to treat fractures of long bones of the body. It comprises a rod which is forced into the medullary canal of a fractured bone and is secured in position by bone screws which extend from the outside of the bone, through the bone itself and through transverse openings defined in the IM nail wherein they are secured in position by suitable means.

It is known to make hollow IM nails from a composite material comprising polyetheretherketone (PEEK) and carbon fibres. However, with such an arrangement, it is difficult to accurately target the transverse openings (especially distal openings) in the nail with the bone screws. As a result, during insertion through the bone and into the transverse openings, the bone screws may not be perfectly axially aligned with the openings and consequently debris may be generated as the screws contact and abrade a region slightly axially offset from perfect axial alignment and/or by friction between screw threads of the bone screws and the walls which define the transverse openings. The debris may comprise small particles of combined PEEK and carbon fibre and may therefore appear as black specks (due to the presence of the black carbon fibre).

The problem of undesirable debris may also arise when the composite material includes glass fibre, basalt fibre or other fibres.

In addition to debris being generated during insertion, in situ movement of the IM nail and associated bone may led to fretting wear at the interface of the IM nail and bone, such wear being most significant in the screw thread/bone contact areas.

Bone plates may comprise contoured and flat plates for spine fixation, orthopaedic fracture applications, humeral, ankle and finger plate systems. In general terms, bone plates may comprise a thin body of suitable shape with transverse openings by which it may be secured in position. If such a plate is made from a composite material comprising PEEK and carbon fibres, debris may be produced during engagement of bone screws with the transverse openings and by fretting wear, as described above in the context of the IM nails.

It is an object of the present invention to address the above-described problems.

It is an object of preferred embodiments of the invention to reduce production of carbon fibre debris during engagement of bone screws with IM nails or bone plates.

According to a first aspect of the invention, there is provided a medical implant, the implant comprising a body which comprises a thermoplastic polymer and at least 10 wt % of a fibrous filler, wherein said body includes a first opening for engagement with a fixing means for fixing the body in position in a human or animal body, wherein said implant includes a protection means which is associated with (e.g. engages) said opening, wherein said protection means comprises a thermoplastic polymer and includes no more than 5 wt % of fibres which have a length of greater than 3 mm.

Said medical implant may comprise an assembly for stabilising a bone in a human or animal body. It may comprise an intra-medullary (IM) nail assembly or a bone plate assembly wherein, suitably, the assemblies comprise an IM nail in combination with said protection means or a bone plate in combination with said protection means respectively. Said medical implant preferably comprises an IM nail assembly.

Said protection means preferably comprises a bio-compatible thermoplastic polymer (hereinafter referred to as “said first polymeric material”). It suitably includes at least 70 wt %, for example 70-96 wt % of said first polymeric material (preferably of a single thermoplastic polymer). Said protection means may include at least 4 wt %, for example 4 to 30 wt %, preferably 6 to 20 wt %, of a radiopaque material. It may include 70 to 96 wt %, preferably 80 to 94 wt % of said first polymeric material. Said radiopaque material may be any material which when added to said first polymeric material of the protection means increases the radiopacity of the combination. Said radiopaque material preferably improves the imageability of the first polymeric material when imaged using both CT and MRI techniques.

Said radiopaque material may comprise a metal, an inorganic material or an iodine-containing organic material.

Said radiopaque material may comprise a metal selected from barium, bismuth, tungsten, gold, titanium, iridium, platinum, rhenium or tantalum; a compound, for example a salt incorporating one of the aforesaid metals; a radiodense salt; or an iodine-containing organic material.

Said radiopaque material preferably has a decomposition temperature which is greater than 300° C., suitably greater than 325° C., preferably greater than 350° C., more preferably greater than 500° C., especially greater than 700° C., suitably so it can be melt-processed with the preferred first polymeric material.

Said radiopaque material preferably comprises a metal selected from those described or a compound for example a salt incorporating one of said metals, provided said compound has a decomposition temperature of greater than 350° C., preferably of greater than 500° C.

Said protection means may include one or a plurality of radiopaque materials. In this case, each radiopaque material may independently be as described herein.

The sum of the wt % of all radiopaque materials in said protection means may be in the range 4 to 30 wt %, suitably 6 to 20 wt %.

The sum of the wt % of all organic polymeric materials and all radiopaque materials in said protection means is suitably at least 80 wt %, preferably at least 90 wt %, more preferably at least 95 wt %, especially at least 99 wt %.

Specific examples of inorganic radiopaque materials include barium sulphate, bismuth compounds (e.g. bismuth oxychloride, bismuth trioxide and bismuth subcarbonate).

Said first polymeric material may be selected from the group comprising self-reinforcing polyphenylene resin (SRP), polyphenylsulphone (PPSU), polysulphone (PSU), polyethersulphone (PES), poly(glycolide (PGA), poly(L-lactide) (PLLA), poly(DL-lactide) (PLDLA), poly(D-lactide) (PDLA), poly(lactic-co-glycolic) acid (PLGA), poly(L-lactide-co-D,L-lactide) (PLDLLA) and poly(e-caprolactone) (PCL).

Said first polymeric material preferably includes a repeat unit of formula

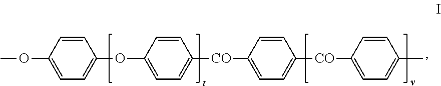

wherein t and v independently represent 0 or 1. Preferred polymeric materials have a said repeat unit wherein either t=1 or v=0; t=0 and v=0; or t=0 and v=1. More preferred have t=1 and v=0; or t=0 and v=0. The most preferred has t=1 and v=0.

Said first polymeric material suitably includes at least 60 mole %, preferably at least 90 mole %, especially at least 98 mole %, of repeat units of formula I. Preferably, said first polymeric material consists essentially of repeat units of formula I. Preferably, said first

In preferred embodiments, said first polymeric material is selected from polyetheretherketone, polyetherketone and polyetherketoneketone. In a more preferred embodiment, said first polymeric material is selected from polyetherketone and polyetheretherketone. In an especially preferred embodiment, said first polymeric material is polyetheretherketone.

Said first polymeric material suitably has a melt viscosity (MV) of at least 0.06 kNsm⁻², preferably has a MV of at least 0.09 kNsm⁻², more preferably at least 0.12 kNsm⁻², especially at least 0.15 kNsm⁻².

MV is suitably measured using capillary rheometry operating at 400° C. at a shear rate of 1000s⁻¹ using a tungsten carbide die, 0.5×3.175 mm.

Said first polymeric material may have a MV of less than 1.00 kNsm⁻², preferably less than 0.5 kNsm⁻².

Said first polymeric material may have a MV in the range 0.09 to 0.5 kNsm⁻², preferably in the range 0.14 to 0.5 kNsm⁻².

Said first polymeric material may have an MV in the range 0.5 to 1.0 kNsm⁻², preferably in the range 0.7 to 1.0 kNsm⁻². MV may be measured by capillary rheometry.

Said first polymeric material may have ISO527 Strength at Break of at least 70 MPa.

Said first polymeric material may have a flexural strength, measured in accordance with

ASTM D790 of at least 145 MPa. The flexural strength is preferably in the range 145-180 MPa, more preferably in the range 145-165 MPa.

Said first polymeric material may have a flexural modulus, measured in accordance with ASTM D790, of at least 2 GPa, preferably at least 3 GPa, more preferably at least 3.5 GPa. The flexural modulus is preferably in the range 3.5-4.5 GPa, more preferably in the range 3.5-4.1 GPa.

Said first polymeric material may have a flexural modulus, measured in accordance with ASTM D790, of at least 7 GPa.

Said protection means is preferably secured relative to, more preferably within, said first opening. It is preferably substantially permanently secured within said first opening. For example, it is preferably arranged not to be disengaged or removed from the opening when a fixing means, for example a screw, is inserted into the opening and engages said protection means. Preferably, said protection means is substantially immovably secured in the opening, preferably so that it does not move significantly when a fixing means is inserted, in use, as described. Thus, said medical implant suitably includes said protection means secured relative to said first opening and such an assembly is separate from any fixing means, for example screw, which may be engaged with said first opening for fixing the body in position.

Said protection means may be secured in said first opening by mechanical, chemical or thermal means. For example, it may be secured in said first opening by being welded or moulded in position by being an interference or push fit within the opening or by mechanical interlocking. Alternatively, it may be secured by chemical/solvent bonding.

Said protection means may line (e.g. cover) at least 30%, preferably at least 50%, more preferably at least 90% of the surface area of the first opening, for example the surface area of a wall which defines a bore of said first opening.

Said first opening in said body preferably extends from one side of the body to an opposite side. It suitably provides a means whereby a fixing means can pass through the body, suitably from one side to the other. Said opening preferably includes a substantially linear portion so that it can receive a fixing means, suitably in the form of an elongate screw.

Said opening preferably has a substantially circular cross-section, at least in part. Said opening may have a length of at least 1 mm and less than 20 mm. For example, in an IM nail, the diameter may be in the range 5-20 mm. For a plate, the thickness may be less than 10 mm, for example in the range 0.5 to 8 mm. Said opening may have a radius of curvature in the range 0.5 to 5 mm. Said fixing means is suitably a bone screw. It may be made from metal or plastics or a combination of the two. Where it is made from plastics, it may suitably be made from preferred first polymeric materials described.

In a first embodiment of the composition of the protection means, said protection means may comprise a radiopaque material in particulate form dispersed within, preferably throughout, said first polymeric material. Said protection means preferably has a substantially constant density throughout. Said composition is preferably substantially homogenous. Suitably, said first polymeric material defines a matrix in which particles of radiopaque material are substantially uniformly dispersed and embedded.

In a preferred example of said first embodiment of the composition, said protection means includes 70 to 96 wt % of said first polymeric material (preferably of formula [I] above, especially polyetheretherketone) and 4 to 30 wt % of radiopaque material (especially particulate material, for example a metal salt such as a barium salt). In an especially preferred example, a protection means includes 80 to 94 wt % of polyetheretherketone and 6 to 20 wt % of a particulate radiopaque material, especially barium sulphate.

In a second embodiment of the composition of the protection means, a wire, for example a metal wire, may be encapsulated in said first polymeric material. The wire may be metal, for example selected from tantalum or another radiopaque metal. In a preferred embodiment, the wire is selected from stainless steel, tungsten and tantalum. Preferably, in the second embodiment, there is provided an elongate wire core (suitably in the form of a spiral) which is coated with said first polymeric material, suitably to substantially fully encapsulate the wire core.

Said protection means preferably includes no more than 3 wt %, more preferably no more than 1 wt % of fibres which have a length greater than 3 mm.

Said protection means suitably includes no more than 5 wt %, preferably no more than 3 wt %, more preferably no more than 1 wt % of carbon fibres which have a length greater than 3 mm.

In some cases, the protection means may be filled with a filler which includes short fibres (e.g. being less than 3 mm in length). In this case, it is found that any abraded particles produced are encapsulated in said thermoplastic polymer which may not be the case when the thermoplastic polymer includes longer fibres.

In one embodiment, said protection means includes no carbon fibres. Said protection means may include no fibre of any description.

In a preferred embodiment, the invention extends to a medical implant, the implant comprising a body which comprises a thermoplastic polymer and at least 10 wt % of a fibrous filler, wherein said body includes a first opening for engagement with a fixing means for fixing the body in position in a human or animal body, wherein said implant includes a protection means which is associated with (e.g. engages) said opening, wherein said protection means comprises a thermoplastic polymer and includes no more than 5 wt % (preferably substantially 0%) of carbon fibres which have a length greater than 3 mm. Other features of the preferred embodiment may be as described herein mutatis mutandis.

Preferably, said protection means consists essentially of a single type of polymeric material selected from said first polymeric materials described (especially polyetheretherketone) and a single type of radiopaque material, which may be barium sulphate.

In a first embodiment of the configuration of said protection means, said protection means suitably provides a lining of the first opening, for example to cover the material (e.g. comprising said thermoplastic polymer and fibrous filler) which defines the first opening. Said protection means suitably defines a bore which is arranged to receive a fixing means, for example a screw, used to secure the body in position. Said protection means may comprise a solid member which includes said bore. The solid member is suitably secured in the first opening. It may be secured therein by mechanical, chemical or thermal means. For example it may be secured by being welded or moulded in position or by being an interference or push fit within the first opening or by mechanical inter-locking. Alternatively, it may be secured by chemical/solvent bonding. Said solid member may include a stepped portion arranged to engage a corresponding stepped portion in said first opening. In a preferred embodiment, said solid member extends from one side of the body to the opposing side, suitably to completely line the opening. Thus, it is preferred that, in use when a fixing means is engaged with the implant, the fixing means only contacts the protection means and does not (and is unable to) contact the material which defines the first opening because it is separated therefrom by said protection means.

Said first embodiment of the first configuration of said protection means may include the first embodiment of the composition of the protection means described above.

In a second embodiment of the configuration of said protection means, said protection means suitably comprises a spacer for spacing the fixing means in use away from the material which defines the first opening, suitably so that, in use, when the fixing means is engaged with the implant, the fixing means only contacts the protection means and does not (and is unable to contact) the material which defines the first opening. Such a spacer may be discontinuous (e.g. it may not define a smooth bore for receiving the fixing means).

In said second embodiment of the configuration, said protection means is preferably resilient. It may be deformable. Said protection means is preferably moveable between a first configuration having a first diameter and a second configuration having a second diameter wherein said first diameter is less than said second diameter. In said implant, said protection means preferably has said second diameter and is urged against the material which defines the first opening. Said protection means may be arranged to have said first diameter to allow insertion of said protection means into said first opening. Suitably, when it has said second diameter, said protection means is retained in position in said first opening.

In said second embodiment of the configuration, said protection means may be in the form of a spiral having its axis about which spirals extend extending axially through the opening from one side of the body to the opposite side. Said spiral may comprise an elongate wire core which is coated with said first polymeric material, as described in accordance with the second embodiment of the composition of the protection means described above

Preferably, in the embodiment described, the protection means is arranged so that, in use, the only contact surfaces of the protection means which initially contact the screw comprise said first polymeric material. Such contact surfaces of the protection means preferably do not include a metal. Thus, the contact surface of the protection means arranged to contact a screw preferably comprise said first polymeric material.

As described, the body of said medical implant comprises a thermoplastic polymer and a fibrous filler. Said thermoplastic polymer (hereinafter referred to as “said second polymeric material”) may be selected from the group comprising self-reinforcing polyphenylene resin (SRP), polyphenylsulphone (PPSU), polysulphone (PSU), polyethersulphone (PES), poly (glycolide (PGA), poly(L-lactide)(PLLA), poly(DL-lactide) (PLDLA), poly(D-lactide)(PDLA), poly (lactic-co-glycolic) acid (PLGA), poly(L-lactide-co-D,L-lactide) (PLDLLA) and poly(e-caprolactone) (PCL).

Said second polymeric material preferably includes a repeat unit of formula

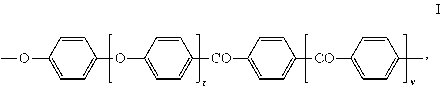

wherein t and v independently represent 0 or 1. Preferred polymeric materials have a said repeat unit wherein either t=1 or v=0; t=0 and v=0; or t=0 and v=1. More preferred have t=1 and v=0; or t=0 and v=0. The most preferred has t=1 and v=0.

Said second polymeric material may independently have any feature of said first polymeric material. Said first and second polymeric materials are preferably substantially identical.

Said fibrous filler may be selected from the group comprising glass, basalt, ceramic, poly(glycolide) (PGA), poly(L-lactide) (PLLA), poly(DL-lactide (PLDLA), poly(D-lactide) (PDLA), poly(lactic-co-glycolic) acid (PLGA), poly(L-lactide-co-D,L-lactide) (PLDLLA) and poly(e-caprolactone) (PCL).

Said fibrous filler is preferably a filler which is coloured, for example, is black and/or which will produce black-containing debris if said body is abraded. Said fibrous filler is preferably carbon-based and more preferably comprises carbon fibre.

Said body preferably includes at least 10 wt % (e.g. at least 30 wt %) of said fibrous filler which includes fibres of length greater than 5 mm (e.g. greater than 10 mm) in length. Preferably, said body includes at least 10 wt % (e.g. at least 30 wt %) of fibres of said fibrous filler which have a length greater than 10 mm.

Said body suitably includes at least 10 wt %, preferably at least 20 wt %, more preferably at least 35 wt %, of carbon fibres which have a length of greater than 10 mm.

Said body may include 25 to 75 wt % (e.g. 40 to 70 wt %) of said fibrous filler (especially carbon fibre) and 25 to 75 wt % (e.g. 30 to 60 wt %) of thermoplastic polymer, especially said second polymeric material described. When said body comprises an IM nail, the first filler may be continuous. In this case, said body may include 45 to 75 wt % of said fibrous filler (e.g. carbon fibre) and 25 to 55 wt % of said second polymeric material (e.g. PEEK). Said body may be hollow. In both cases, the fibrous filler preferably has an average length of greater than 10 mm.

Said body may alternatively comprise a bone plate. In this case, the fibrous filler may be discontinuous; and said body may include 25 to 50 wt % of said fibrous filler (e.g. discontinuous carbon fibre) and 50 to 75 wt % of said second polymeric material (e.g. PEEK). In this case, the body may not be hollow but may be solid. The fibrous filler may have an average length of at least 5 mm, for example at least 10 mm.

Said body of said medical implant may include a second opening so that said body includes at least two openings for engagement with respective fixing means, wherein said two openings are spaced apart. In the case of an IM nail, the openings may define proximal and distal openings in the nail. Said second opening may independently have any feature of said first opening. Said first and second openings are preferably substantially identical.

Said first opening is preferably a transversely-extending opening. Thus, it suitably extends transverse to the elongate extent of an IM nail; or transverse to a main plane of the bone plate.

Said second opening is preferably a transversely-extending opening. Thus, it suitably extends transverse to the elongate extent of an IM nail; or transverse to a main plane of the bone plate.

The invention extends in a second aspect to a kit comprising a medical implant as described and a fixing means for fixing the body of the implant in position. Said implant and fixing means are preferably sterile. One or both of the implant and/or fixing means may be provided in a receptacle in a sterile state.

The invention extends in a third aspect to a medical implant of the first aspect in combination with a fixing means (preferably a plurality of fixing means), wherein said fixing means extends through the first opening.

The invention extends in a fourth aspect to a protection means for a body, for example IM nail or plate, said protection means being as described according to the first aspect.

The invention extends, in a fifth aspect, to a method of assembling a medical implant as described in the first aspect, the method comprising:
(i) selecting a body which comprises at least 10 wt % of a fibrous filler, wherein said body includes a first opening for engagement with a fixing means for fixing the body in position in a human or animal body;
(ii) selecting a protection means which comprise a thermoplastic polymer and includes no more than 5 wt % of fibres which have a length of greater than 3 mm;
(iii) securing said protection means within said first opening, preferably so said protection means lines a mouth of the first opening and/or lines at least 50% (preferably at least 90%) of the surface area of the first opening.

The invention extends, in a sixth aspect, to a method of stabilising and/or supporting a bone in a human or animal body, the method comprising:
(i) selecting a medical implant as described in said first aspect;
(ii) contacting said medical implant with a bone in a human or animal body;
(iii) selecting fixing means;
(iv) engaging said fixing means in the first opening in the body of the medial implant thereby to secure the body in position wherein it stabilises and/or supports the bone.

The invention extends, in a seventh aspect, to the use of a medical implant as described in the first aspect for stabilising and/or supporting a bone in a human or animal body.

Any invention described herein may be combined with any feature of any other invention described herein mutatis mutandis.

Specific embodiments of the invention will now be described, by way of example, with reference to the accompanying drawings in which:

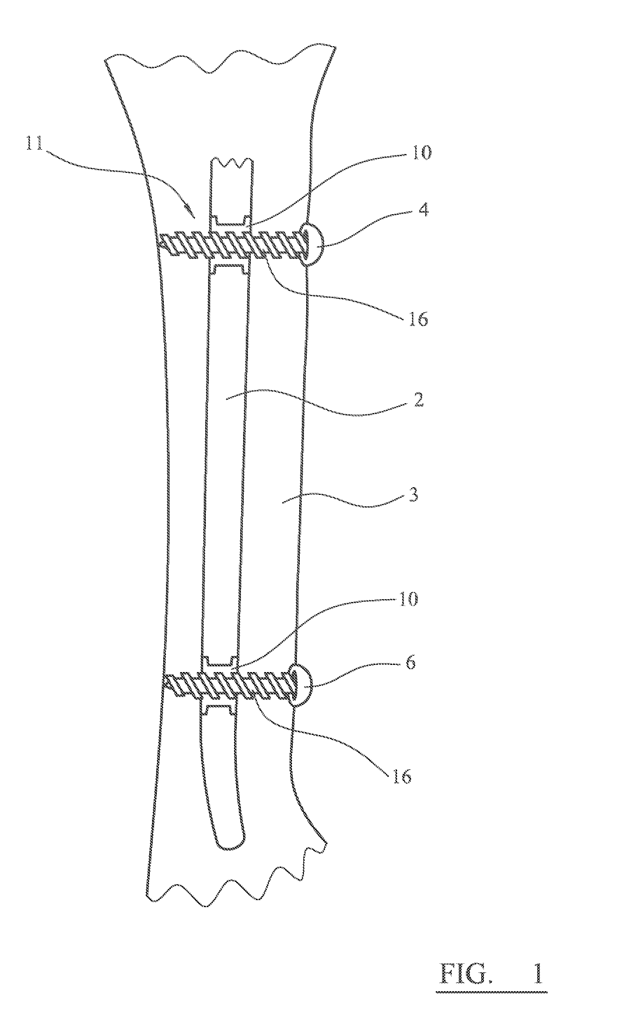

FIG. 1 is a cross-section through part of a bone showing an intra-medullary (IM) nail fixed in position by bone screws;

FIG. 2 is a cross-section through part of the IM nail of FIG. 1;

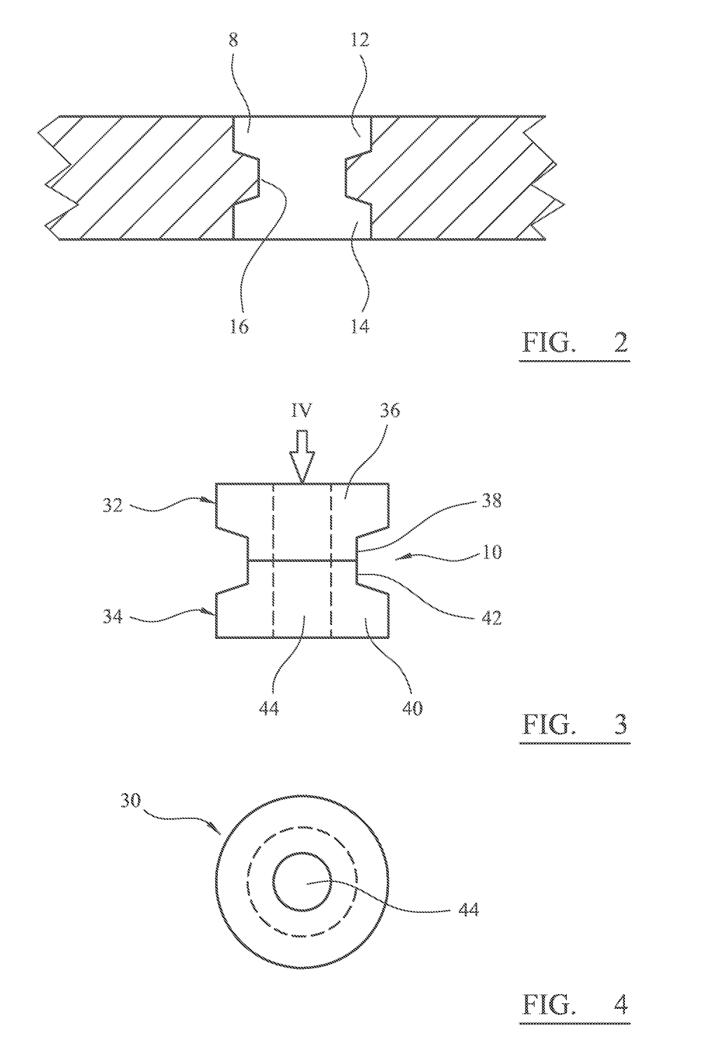

FIG. 3 is a side view of an insert assembly for engagement with the IM nail of FIG. 3;

FIG. 4 is a view of the assembly of FIG. 3 in the direction of arrow IV;

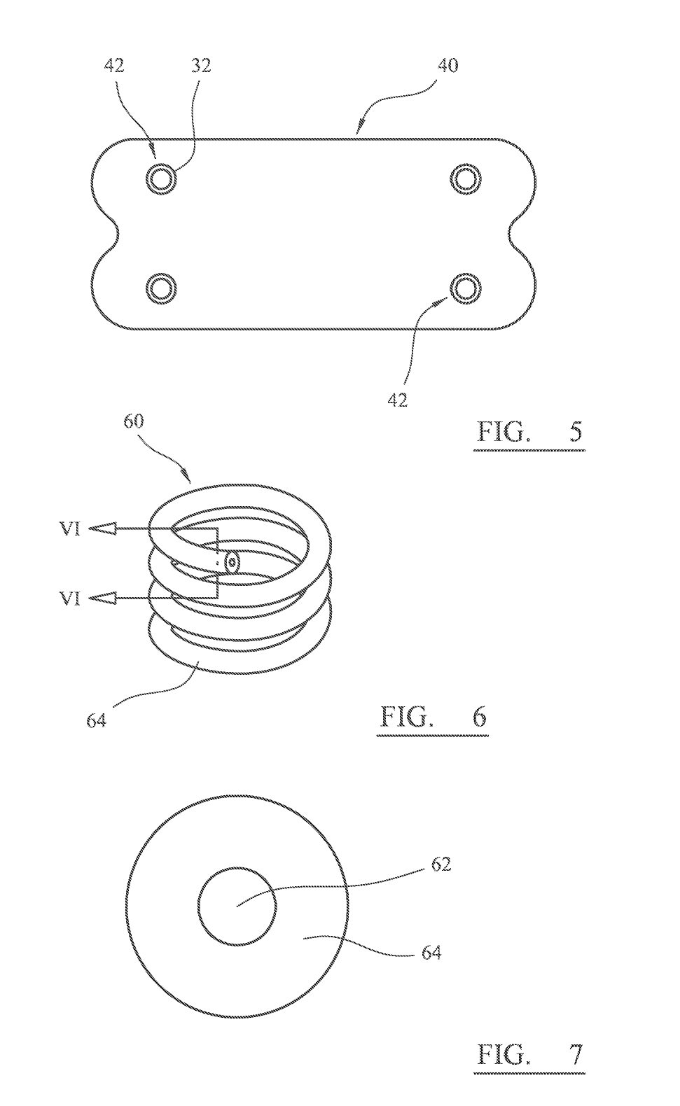

FIG. 5 is a plan view of a bone plate assembly;

FIG. 6 is a perspective view of an alternative insert assembly for an IM nail; and

FIG. 7 is a cross-section along line VI-VI of FIG. 6.

Referring to FIG. 1, an IM nail 2 is positioned within a medullary canal 3 and secured in position by bone screws 4, 6. The nail 2 includes bores 8 (FIG. 2) which engage insert assemblies 10 to define an IM nail assembly 11. The insert assemblies engage the screws 4, 6. The bores 8 include respective mouths 12, 14 (FIG. 2) separated by a narrower portion 16. Each bore is arranged to engage a respective insert assembly 10 (shown in detail in FIGS. 3 and 4) which corresponds in shape to that of the bore 8 with which it is engaged. The insert assembly 10 as shown in FIG. 3 is in two parts, 32, 34. Part 32 includes a head 36 and a narrower neck 38; and similarly, part 34 includes a head 40 and a narrower neck 42. A bore 44 extends through the assembly from one side to the other.

Part 32 of the assembly 10 is engaged with a nail by insertion into the nail via mouth 12; and part 34 is engaged with the nail by insertion into the nail via mouth 14. When in position the parts 32, 34 abut one another and define bore 44 which can receive screws 4, 6.

In one embodiment parts 32, 34 may be spin welded (for symmetrical assemblies) within respective mouths 12, 14; may be an interference fit therewithin; may be adhered in position; or may be secured by welding, for example by ultrasonic, induction, laser or thermal welding. Alternatively, solvent or chemical bonding may be used to secure parts 32, 34 in position. Alternatively, in each case, the insert assembly 10, defined by parts 32, 34, lines the bore 8.

The nail 2 is suitably hollow (although this is not depicted in the figures in the interests of clarity) and is made from a polyetheretherketone (PEEK)/carbon fibre composite by known means. Mouths 12, 14 and narrower portion 16 are suitably defined in the nail during manufacture. Parts 32, 34 of the insert assemblies 10 are produced separately from the nail. They may be produced by injection moulding a polymeric composition comprising PEEK and barium sulphate. After manufacture, the IM nail and insert assemblies may be engaged to define an IM nail assembly.

As described, the IM nail is made from a composite material comprising polyetheretherketone and carbon fibre. Such a material is very stiff and strong. In contrast, the inserts assemblies 20 are made from PEEK and barium sulphate. The screws 4, 6 are suitably made from titanium, although they may be made from other materials.

The IM nail assembly 11, including IM nail 2 and parts 32, 34 is introduced into a medullary canal in a conventional manner to stabilise a fractured bone. Then, screws 4, 6 are used to secure the nail in position. Since parts 32, 34 incorporate barium sulphate, a radiopaque material, the parts are visible under X-rays. In contrast, since the nail 2 comprises PEEK and carbon fibre it is generally not readily visible under X-rays. By including parts 32, 34, it is possible for a surgeon to view the opening (e.g. the axis and location) into which a screw 4, 6 is to be inserted and to accurately position the screw. Furthermore, should a screw 4, 6 be slightly misaligned as it is contacted with the nail assembly, the screw will contact an associated part 32, 34 of an insert assembly. Consequently, any abraded particles generated will comprise PEEK and barium sulphate rather than black carbon fibre (since nail 2 itself should not be contacted). Thus, use of parts 32, 34 minimises and/or eliminates generation and/or release of black carbon fibre particles from the IM nail assembly.

Referring to FIG. 5, the bone plate assembly 40 comprises a bone plate (which may be of any known shape and/or size) which includes openings 42. The openings have a cross-section as for the opening shown in FIG. 2 and include an insert assembly 10 (which is substantially as described in FIGS. 1 to 4) positioned within a recess (not shown) which is as described in FIGS. 1 to 4. The advantages in using the assembly in terms of accurately positioning screws in the bone plate via openings 32 and minimal or zero release of carbon fibre-containing debris may be as described for the IM nail assembly.

An alternative insert 60 for an IM nail 2 is shown in FIGS. 6 and 7. The insert 60 is in the form of a wire spiral which includes a tantalum core 62 coated with a PEEK layer 64 which provides a sheath for the core. The insert 60 is arranged to be wound (mechanically prepared) to define a reduced diameter “loaded” state. It may then be introduced into a bore in an IM nail to define an IM nail assembly. The bore may be parallel sided and/or may include a step, for example at one end, to facilitate insertion and/or retention of the insert 60 within the bore. After introduction into the bore, the insert expands radially to fill the bore and become an interference fit therewithin. Advantageously, the tantalum core is visible under X-rays and so can effectively mark the opening into which screws (e.g. 4 or 6) may be inserted to facilitate accurate positioning of the screws. In the case of misalignment of a screw during its insertion, the misaligned screw will contact the PEEK coating (which does not contain any carbon fibre) so only PEEK particles will be released.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1. A medical implant, the implant comprising a body which comprises a thermoplastic polymer and at least 10 wt % of a fibrous filler, wherein said body includes a first opening for engagement with a fixing means for fixing the body in position in a human or animal body, wherein said implant includes a protection means which is associated with (e.g. engages) said opening, wherein said protection means comprises a thermoplastic polymer and includes no more than 5 wt % of fibres which have a length of greater than 3 mm.

2. An implant according to claim 1, wherein said implant comprises an assembly for stabilising a bone in a human or animal body.

3. An implant according to claim 1, wherein said implant is selected from an intra-medullary nail assembly and a bone plate assembly.

4. Any implant according to claim 1, wherein said protection means includes at least 70 wt % of a first polymeric material and at least 4 wt % of a radiopaque material.

5. An implant according to claim 1, wherein said protection means includes a first polymeric material which includes a repeat unit of formula 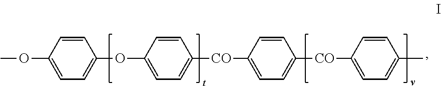
wherein t and v independently represent 0 or 1.

6. An implant according to claim 5, wherein t=1 and v=0.

7. An implant according to claim 5, wherein said first polymeric material includes at least 90 mol% of repeat units of formula I.

8. An implant according to claim 1, wherein said thermoplastic polymer of said protection means is polyetheretherketone.

9. An implant according to claim 1, wherein said protection means is secured relative to said first opening.

10. An implant according to claim 1, wherein said protection means is substantially immovably secured in the opening.

11. An implant according to claim 1, wherein said first opening in said body extends from one side of the body to an opposite side.

12. An implant according to claim 1, wherein said protection means includes less than 3 wt % of fibres which have a length greater than 3 mm.

13. An implant according to claim 1, wherein said protection means includes less than 5 wt % of carbon fibres which have a length greater than 3 mm.

14. An implant according to claim 1, wherein said protection means includes no carbon fibres.

15. An implant according to claim 1, wherein said protection means provides a lining of the first opening.

16. An implant according to claim 1, wherein said protection means comprises a spacer for spacing the fixing means in use away from the material which defines the first opening.

17. An implant according to claim 1, wherein the body of said medical implant comprises a thermoplastic polymer and a fibrous filler, wherein said thermoplastic polymer includes a repeat unit of formula 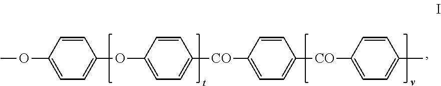
wherein t and v independently represent 0 or 1.

18. An implant according to claim 1, wherein said fibrous filler of said body is carbon-based.

19. An implant according to claim 1, wherein said body includes at least 30 wt % of said fibrous filler which includes fibres of length greater than 5 mm.

20. An implant according to claim 1, wherein said body includes 25 to 75 wt % of said fibrous filler and 25 to 75 wt % of thermoplastic polymer.

21. An implant according to claim 1, wherein said body is an intra-medullar nail and said first filler is continuous.

22. An implant according to claim 1, wherein the fibrous filler is discontinuous.

23. A kit comprising a medical implant according to claim 1 and a fixing means for fixing the body of the implant in position.

24. A method of assembling a medical implant according to claim 1, the method comprising:
(i) selecting a body which comprises at least 10 wt % of a fibrous filler, wherein said body includes a first opening for engagement with a fixing means for fixing the body in position in a human or animal body;
(ii) selecting a protection means which comprise a thermoplastic polymer and includes no more than 5 wt % of fibres which have a length of greater than 3 mm;
(iii) securing said protection means within said first opening, preferably so said protection means lines a mouth of the first opening and/or lines at least 50% (preferably at least 90%) of the surface area of the first opening.
